(19) United States
(12) Patent Application Publication
(10) Pub. No.: US 2008/0254114 A1
(43) Pub. Date: Oct. 16, 2008

(54) Controlled Release Compositions Comprising Heterocyclic Amide Derivative Nanoparticles

(75) Inventors: Scott A. Jenkins, Downingtown, PA (US); Gary Liversidge, West Chester, PA (US)

Correspondence Address: Fox Rothschild, LLP;Elan Pharma International Limited, 2000 Market Street, Philadelphia, PA, 19103, US

(73) Assignee: Elan Corporation plc, Dublin 2 (IE)

(21) Appl. No.: 11/571,377

(22) PCT Filed: Mar. 2, 2006
(86) PCT No.: PCT/US2006/007465

§ 371 Date: Jan. 14, 2008

Related U.S. Application Data
(60) Provisional application No. 60/658,259, filed on Mar. 3, 2005.

(51) Int. Cl. A61K 9/16; A61K 31/404; A61K 9/54; A61K 9/26; A61P 11/06

(52) U.S. Cl. 424/456; 424/489; 514/415; 424/490; 424/469

(57) ABSTRACT

The present invention is directed to compositions comprising nanoparticulate heterocyclic amide derivative and preferably zafirlukast nanoparticles, also collectively referred to as “active ingredient,” having improved solubility in water. The nanoparticles of the composition have an effective average particle size of less than about 2,000 nm, and are useful in the treatment of asthma. The invention also relates to a multiparticulate modified release composition comprising the active ingredient that in operation delivers the drug in a pulsed or bimodal manner for the treatment of asthma. The controlled release composition comprises an immediate release component and a modified release component. The immediate release component comprises a first population of heterocyclic amide derivative, and preferably zafirlukast particles, and the modified release component comprises a second population of heterocyclic amide derivative, and preferably zafirlukast nanoparticles, and a controlled release component, wherein the combination of the immediate release and modified release components in operation delivers the active ingredient in a pulsed or bimodal manner. The heterocyclic amide derivative can be released from the multiparticulate particles in an erosable, diffusion or osmotic controlled release system.

### FIELD OF THE INVENTION

The present invention relates to a controlled release composition comprising a nanoparticulate heterocyclic amide derivative and preferably zafirlukast nanoparticles for use in the treatment of patients suffering from asthma. The nanoparticles have an effective average-particle size of less than about 2,000 nm.

### BACKGROUND OF THE INVENTION

### A. Background Regarding Nanoparticulate Compositions

Nanoparticulate compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer. The '684 patent does not describe nanoparticulate compositions of zafirlukast.

Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” and 6,432,381 for “Methods for Targeting Drug Delivery to the Upper and/or Lower Gastrointestinal Tract,” all of which are specifically incorporated by reference. In addition, United States Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

### B. Background Regarding Zafirlukast

Zafirlukast is a synthetic, selective peptide leukotriene receptor antagonist (LTRA), with the chemical name 4-(5-cyclopentyloxy-carbonylamino-1-methyl-indol-3-ylmethyl)-3-methoxy-N-o-tolylsulfonylbenzamide. The molecular weight of zafirlukast is 575.7. Zafirlukast is marketed under the registered trademark ACCOLATE by AtraZeneca Pharmaceuticals, LP, of Wilmington, Del.

The empirical formula is: C₃₁H₃₃N₃O₆S.

Zafirlukast, a fine white to pale yellow amorphous powder, is practically insoluble in water. It is slightly soluble in methanol and freely soluble in tetrahydrofuran, dimethyl-sulfoxide, and acetone.

ACCOLATE® is supplied as 10 and 20 mg tablets for oral administration.

Film-coated tablets contain croscarmellose sodium, lactose, magnesium stearate, microcrystalline cellulose, povidone, hydroxypropylmethylcellulose, and titanium dioxide.

Zafirlukast is a selective and competitive receptor antagonist of leukotriene D₄ and E₄ (LTD₄ and LTE₄), components of slow-reacting substance of anaphylaxis (SRSA). Cysteinyl leukotriene production and receptor occupation have been correlated with the pathophysiology of asthma, including airway edema, smooth muscle constriction, and altered cellular activity associated with the inflammatory process, which contribute to the signs and symptons of asthma. Patients with asthma were found in one study to be 25-100 times more sensitive to the bronchoconstricting activity of inhaled LTD₄ than nonasthmatic subjects.

In vitro studies demonstrated that zafirlukast antagonized the contractile activity of three leukotrienes (LTC₄, LTD₄ and LTE₄) in conducting airway smooth muscle from laboratory animals and humans. Zafirlukast prevented intradermal LTD₄-induced increases in cutaneous vascular permeability and inhibited inhaled LTD₄-induced influx of eosinophils into animal lungs. Inhalational challenge studies in sensitized sheep showed that zafirlukast suppressed the airway responses to antigen; this included both the early- and late-phase response and the nonspecific hyperresponsiveness.

In humans, zafirlukast inhibited bronchoconstriction is caused by several kinds of inhalational challenges. Pretreatment with single oral doses of zafirlukast inhibited the bronchoconstruction caused by sulfur dioxide and cold air in patients with asthma. Pretreatment with single doses of zafirlukast attenuated the early- and late-phase reaction caused by inhalation of various antigens such as grass, cat dander, ragweed, and mixed antigens in patients with asthma. Zafirlukast also attenuated the increase in bronchial hyperresponsiveness to inhaled histamine that followed inhaled allergen challenge.

Zafirlukast is rapidly absorbed following oral administration. Peak plasma concentrations are generally achieved three hours after oral administration. The absolute bioavailability of zafirlukast is unknown. In two separate studies, one using a high fat and the other a high protein meal, administration of zafirlukast with food reduced the mean bioavailability by approximately 40%. Physicians Desk Reference, 58^{th} Edition (2004), p. 651.

U.S. Pat. No. 4,859,692 to Bernstein et al., is for “heterocyclic amide derivatives and pharmaceutical use.” U.S. Pat. No. 5,294,636 to Edwards et al. is for “crystalline form of indole derivative and pharmaceutical method thereof.” U.S. Pat. Nos. 5,319,097 to Holohan et al. is for “pharmaceutical agents.” U.S. Pat. No. 5,482,963, also to Holohan et al., is for “pharmaceutical agents useful in leukotriene antagonists.” U.S. Pat. No. 5,583,152 to Bernstein et al. is for a “method for treating vasopastic cardiovascular diseases heterocyclic amide derivatives.” U.S. Pat. No. 5,612,367 to Timko et al. is for a “method of enhancing bioavailability of pharmaceutical agents. Finally, U.S. Pat. No. 6,143,775, also to Holohan et al., is for a “process for preparing pharmaceutical composition containing a heterocyclic amide.”

Due to the drug's high degree of bioavailability and rapid metabolism, it would be advantageous to provide heterocyclic amide derivative nanoparticles, preferably nanoparticulate zafirlukast, with a drug delivery formulation that releases the active in a controlled or delayed release profile. More specifically, it would be a tremendous benefit to patients suffering from asthma if the drug could be formulated to be released in a two phase or pulsatile manner so that the drug can provide its pharmacological activity over an extended period of time, in particular, over a twenty-four hour period. In this manner, patients suffering from asthma can benefit from the drug's therapeutic effects for extended periods of time without the need to take more than one dosage per day.

Because zafirlukast is practically insoluble in water, significant bioavailability can be problematic. There is a need in the art for nanoparticulate zafirlukast formulations which overcome this and other problems associated with prior conventional zafirlukast formulations. The present invention satisfies this need.

### SUMMARY OF THE INVENTION

It is an object of the present invention to provide a controlled release composition containing nanoparticulate heterocyclic amide derivatives, and preferably zafirlukast nanoparticles, which in operation produces a plasma profile substantially similar to the plasma profile produced by the administration of two or more IR dosage forms given sequentially.

It is a further object of the invention to provide a controlled release composition which in operation delivers the nanoparticulate heterocyclic amide derivative, and preferably zafirlukast nanoparticles, in a pulsatile manner.

Another object of the invention is to provide a controlled release composition which substantially mimics the pharmacological and therapeutic effects produced by the administration of two or more IR dosage forms given sequentially.

Another object of the present invention is to provide a controlled release composition which substantially reduces or eliminates the development of patient tolerance to the heterocyclic amide derivative nanoparticles, preferably nanoparticulate zafirlukast of the composition.

Another object of the invention is to provide a controlled release composition in which a first portion of the active ingredient, i.e., the heterocyclic amide derivative nanoparticles, preferably nanoparticulate zafirlukast, is released immediately upon administration and a second portion of the active ingredient is released rapidly after an initial delay period in a bimodal manner.

Another object of the present invention is to formulate the dosage in the form of erodable formulations, diffusion controlled formulations or osmotic controlled formulations.

Another object of the invention is to provide a controlled release composition capable of releasing the nanoparticulate heterocyclic amide derivative, and preferably zafirlukast nanoparticles, in a bimodal or multi-modal manner in which a first portion of the active is released either immediately or after a delay time to provide a pulse of drug release, and one or more additional portions of the nanoparticulate heterocyclic amide derivative, and preferably zafirlukast nanoparticles, is released, each after a respective lag time, to provide additional pulses of drug release during a period of up to twenty-four hours.

Another object of the invention is to provide solid oral dosage forms comprising a controlled release composition comprising zafirlukast.

Other objects of the invention include provision of a once daily dosage form of zafirlukast which, in operation, produces a plasma profile substantially similar to the plasma profile produced by the administration of two immediate release dosage forms given sequentially and a method for treatment of asthma based on the administration of such a dosage form.

The above objects are realized by a controlled release composition having a first component comprising a first population of nanoparticulate heterocyclic amide, preferably zafirlukast nanoparticles, and a second component or formulation comprising a second population of nanoparticulate heterocyclic amide, preferably zafirlukast nanoparticles. The ingredient-containing particles of the second component further comprises a modified release constituent comprising a release coating or release matrix material, or both. Following oral delivery, the composition in operation delivers the heterocyclic amide derivative nanoparticles, and preferably nanoparticulate zafirlukast, in a pulsatile manner.

The present invention utilizes controlled release delivery of nanoparticulate heterocyclic amide, preferably zafirlukast nanoparticles, from a solid oral dosage formulation to allow dosage less frequently than before, and preferably once-a-day administration, increasing patient convenience and compliance. The mechanism of controlled release would preferably utilize, but not be limited to, erodable formulations, diffusion controlled formulations and osmotic controlled formulations. A portion of the total dose may be released immediately to allow for rapid onset of effect. The invention would be useful in improving compliance and, therefore, therapeutic outcome for all treatments requiring zafirlukast, including but not limited to, treatment of asthma. This approach would replace conventional zafirlukast tablets and solution, which are administered twice a day as adjunctive therapy in the treatment of asthma.

The present invention also relates to a controlled modified release composition for the controlled release of nanoparticulate heterocyclic amide, preferably zafirlukast nanoparticles. In particular, the present invention relates to a controlled release composition that in operation delivers heterocyclic amide derivative nanoparticles, and preferably nanoparticulate zafirlukast, in a pulsatile manner, preferably during a period of up to twenty-four hours. The present invention further relates to solid oral dosage forms containing a controlled release composition.

Preferred controlled release formulations are erodable formulations, diffusion controlled formulations and osmotic controlled formulations. According to the invention, a portion of the total dose may be released immediately to allow for rapid onset of effect, with the remaining portion of the total dose released over an extended time period. The invention would be useful in improving compliance and, therefore, therapeutic outcome for all treatments requiring zafirlukast, including but not limited to, the treatment of asthma.

The present invention relates to nanoparticulate compositions comprising an heterocyclic amide derivative, preferably zafirlukast. The compositions comprise nanoparticulate zafirlukast particles, and at least one surface stabilizer adsorbed on the surface of the zafirlukast particles. The nanoparticulate zafirlukast particles have an effective average particle size of less than about 2,000 nm.

A preferred dosage form of the invention is a solid dosage form, although any pharmaceutically acceptable dosage form can be utilized.

Another aspect of the invention is directed to pharmaceutical compositions comprising a nanoparticulate heterocyclic amide derivative, preferably zafirlukast nanoparticles and at least one surface stabilizer, a pharmaceutically acceptable carrier, as well as any desired excipients.

Another aspect of the invention is directed to a nanoparticulate heterocyclic amide derivative, preferably a nanoparticulate zafirlukast composition, having improved pharmacokinetic profiles as compared to conventional zafirlukast formulations.

Another embodiment of the invention is directed to nanoparticulate zafirlukast compositions comprising one or more additional compounds useful in the treatment of asthma.

This invention further discloses a method of making the inventive nanoparticulate zafirlukast composition. Such a method comprises contacting the nanoparticulate zafirlukast with at least one surface stabilizer for a time and under conditions sufficient to provide a stabilized nanoparticulate zafirlukast composition.

The present invention is also directed to methods of treatment including but not limited to, the treatment of asthma using the novel nanoparticulate zafirlukast compositions disclosed herein. Such methods comprise administering to a subject a therapeutically effective amount of a nanoparticulate heterocyclic amide derivative, preferably, zafirlukast. Other methods of treatment using the nanoparticulate compositions of the invention are known to those of skill in the art.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

Controlled release compositions similar to those disclosed herein are disclosed and claimed in the U.S. Pat. Nos. 6,228,398 and 6,730,325 to Devane et al., both of which are incorporated by reference herein.

U.S. Provisional Application No. 60/638,826, filed Dec. 22, 2004, entitled “Nanoparticulate Bicalutamide Formulations” is also specifically incorporated by reference herein.

U.S. Provisional Application No. 60/641,916, filed Jan. 6, 2005, entitled “Nanoparticulate Candersartan Cilexetil Formulations” is also specifically incorporated by reference herein.

U.S. Provisional Application No. 60/643,725, filed Jan. 12, 2005, entitled “Controlled Release Compositions Comprising An Acylanilide” is also specifically incorporated by reference herein.

U.S. Provisional Application No. 60/647,311, filed Jan. 26, 2005, entitled “A Controlled Release Oral Dosage Formulation of Seroquec” is also specifically incorporated by reference herein.

U.S. Provisional Application No. ______, filed Feb. 15, 2005, entitled “Aerosol and Injectable Formulations of Nanoparticulate Benzodiazepine” is also specifically incorporated by reference herein.

U.S. Provisional Application No. ______, filed Feb. 16, 2005, entitled “Controlled Release Compositions Comprising Levetiracetam” also specifically incorporated by reference herein.

U.S. Provisional Application No. ______, filed Feb. 24, 2005, entitled “Injectable Formulations of a Nanoparticulate Taxoid,” is also specifically incorporated by reference herein.

In a preferred embodiment of a multiparticulate modified release composition according to the invention the first component includes an immediate release constituent.

In the second component, the modified release coating applied to the second population or presence of a modified release matrix material in the second population of nanoparticulate heterocyclic amide derivative, and preferably zafirlukast nanoparticles, causes a lag time between the release of zafirlukast from the first population of zafirlukast particles and the release of active ingredient from the second population of active ingredient containing particles. The duration of the lag time may be varied by altering the composition and/or the amount of the modified release coating and/or altering the composition and/or amount of modified release matrix material utilized in the composition or formulation. Preferred types of formulations for use in varying the lag time are erodable formulations, diffusion controlled formulations and osmotic controlled formulations. Thus, the duration of the lag time can be designed to mimic a desired plasma profile.

### Erodable Formulations

The subsequent formulations can be in the form of erodable formulations in which the active ingredients and modified release constituent consisting of at least one of modified release coatings and modified release matrix materials would dissolve in water, over time losing their structural integrity. One manner in which this could occur would be that the active ingredients and modified release coatings and/or matrix materials would dissolve after human ingestion over a controlled period of time.

### Diffusion Controlled Formulations

The subsequent formulations can be in the form of diffusion controlled formulations which would allow the gradual spread of the subsequent population of particles to scatter or spread out in a liquid medium, are referenced, for example, in U.S. Pat. No. 6,586,006 to Roser et al., which is incorporated by reference herein.

### Osmotic Controlled Formulations

Controlled release of the subsequent formulations could be controlled by osmosis. U.S. Pat. No. 6,110,498 to Rudnic et al. for an “osmotic drug delivery system” discloses a system which dispenses a therapeutic agent having limited water solubility in solubilized form. The delivery system comprises a core that is free of swellable polymers and comprises nonswelling solubilizing agents and wicking agents. The solubilized therapeutic agent is delivered through a passageway in the semipermeable coating of the tablet.

U.S. Pat. No. 6,814,979 B2 also to Rudnic et al. describes an osmotic pharmaceutical delivery system comprising (a) a semi-permeable wall that maintains its integrity during pharmaceutical delivery and which has at least one passage therethrough; (b) a single, homogeneous composition within said wall, which composition consists essentially of (i) a pharmaceutically active agent, (ii) at least one non-swelling solubilizing agent which enhances the solubility of the pharmaceutically active agent; (iii) at least one non-swelling osmotic agent and (iv) a non-swelling wicking agent dispersed throughout the composition which enhances the surface area contact of the pharmaceutical agent with the incoming aqueous fluid. Both of these patents to Rudnic et al. are incorporated by reference herein.

The present invention is also directed to nanoparticulate compositions comprising an heterocyclic amide derivative, preferably zafirlukast. The compositions comprise nanoparticulate zafirlukast particles and preferably at least one surface stabilizer adsorbed on the surface of the drug. The nanoparticulate heterocyclic amide derivative, preferably zafirlukast, particles have an effective average particle size of less than about 2,000 nm.

As taught in the '684 patent, not every combination of surface stabilizer and active agent will result in a stable nanoparticulate composition. It was surprisingly discovered that stable, nanoparticulate heterocyclic amide derivative, preferably zafirlukast, formulations can be made.

Advantages of the nanoparticulate heterocyclic amide derivative, preferably zafirlukast, formulations of the invention include, but are not limited to: (1) smaller tablet or other solid dosage form size; (2) smaller doses of drug required to obtain the same pharmacological effect as compared to conventional forms of zafirlukast; (3) increased bioavailability as compared to conventional forms of zafirlukast; (4) improved pharmacokinetic profiles; (5) improved bioequivalency of the nanoparticulate zafirlukast compositions; (6) an increased rate of dissolution for the nanoparticulate zafirlukast compositions as compared to conventional forms of the same active compound; (7) bioadhesive zafirlukast compositions; and (8) the nanoparticulate heterocyclic amide derivative, preferably zafirlukast compositions can be used in conjunction with other active agents useful for the treatment of asthma.

The present invention also includes nanoparticulate heterocyclic amide derivatives, preferably zafirlukast compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable heterocyclic amide derivative, preferably zafirlukast particles, “stable” means that the particles do not appreciably flocculate or agglomerate due to interparticle attractive forces or otherwise spontaneously increase in particle size.

### A. Preferred Characteristics of the Zafirlukast Compositions of the Invention

### 1. Increased Bioavailability

The heterocyclic amide derivative, preferably zafirlukast formulations of the invention are proposed to exhibit increased bioavailability and require smaller doses as compared to prior conventional heterocyclic amide derivative, preferably zafirlukast formulations.

### 2. Dissolution Profiles of the Nanoparticulate Zafirlukast Compositions of the Invention

The heterocyclic amide derivative, preferably zafirlukast compositions of the invention are proposed to have unexpectedly dramatic dissolution profiles. Rapid dissolution of an administered active agent is preferable, as faster dissolution generally leads to faster onset of action and greater bioavailability. To improve the dissolution profile and bioavailability of the heterocyclic amide derivative, in particular, the zafirlukast active compound, it would be useful to increase zafirlukast's dissolution so that it could attain a level close to 100%.

The heterocyclic amide derivative, preferably the nanoparticulate zafirlukast compositions of the invention, preferably have a dissolution profile in which within about 5 minutes at least about 20% of the composition is dissolved. In other embodiments of the invention, at least about 30% or about 40% of the naonoparticulate zafirlukast composition is dissolved within about 5 minutes. In yet other embodiments of the invention, preferably at least about 40%, about 50%, about 60%, about 70%, or about 80% of the nanoparticulate zafirlukast composition is dissolved within about 10 minutes. Finally, in another embodiment of the invention, preferably at least about 70%, about 80%, about 90%, or about 100% of the stabilized nanoparticulate zafirlukast composition is dissolved within about 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

3. Modified Zafirlukast Compositions Including Compositions Used in Conjunction with Other Active Agents

Conventional zafirlukast tablets have limited bioavailability because zafirlukast is practically insoluble in water. The present invention is proposed to comprise stabilized nanoparticulate zafirlukast compositions to improve the dissolution rate of the practically insoluble active compound. The improvement in dissolution rate is proposed to enhance the bioavailability of zafirlukast, allowing a smaller dose to give the same in vivo blood levels as larger dosage amounts required in the past. In addition, the enhanced dissolution rate is proposed to allow for a larger dose to be absorbed, which increases the efficacy of zafirlukast and therefore, therapeutic outcome for all treatments requiring zafirlukast, including, but not limited to, the treatment of asthma.

Another embodiment of the invention is directed to an heterocyclic amide derivative, preferably zafirlukast compositions comprising one or more compounds for use in the treatment of asthma.

### B. Compositions

The present invention provides compositions comprising nanoparticulate heterocyclic amide derivatives, and preferably zafirlukast nanoparticles, and at least one surface stabilizer. The surface stabilizers preferably are adsorbed on, or associated with, the surface of the heterocyclic amide derivative, preferably zafirlukast particles. Surface stabilizers especially useful herein preferably physically adhere on, or associate with, the surface of the nanoparticulate zafirlukast particles but do not chemically react with the zafirlukast particles or themselves. Individually adsorbed molecules of the surface stabilizer are essentially free of intermolecular cross-linkages.

The present invention also includes heterocyclic amide derivative and preferably zafirlukast compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like.

### 1. Surface Stabilizers

The choice of a surface stabilizer for an heterocyclic amide derivative, and preferably zafirlukast, is non-trivial and required extensive experimentation to realize a desirable formulation. Accordingly, the present invention is directed to the surprising discovery that stabilized nanoparticulate zafirlukast compositions can be made that will not agglomerate or adhere to one another.

Combinations of more than one surface stabilizer can be used in the invention. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® products such as e.g., Tween® 20 and Tween® 80 (ICI Speciality Chemicals)); polyethylene glycols (e.g., Carbowax® 3550 and 934 (Union Carbide)), polyoxy-ethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronic® F68 and F108, which are block copolymers of ethylene oxide and propylene oxide); poloxamines (e.g., Tetronic® 908, also known as Poloxaminem 908, which is a tetrafunctional block copolymer derived from sequential addition of propylene oxide and ethylene oxide to ethylenediamine (BASF Wyandotte Corporation, Parsippany, N.J.)); Tetronic® 1508 (T-1508) (BASF Wyandotte Corporation), Triton® X-200, which is an alkyl aryl polyether sulfonate (Rohm and Haas); Crodestas™ F-110, which is a mixture of sucrose stearate and sucrose distearate (Croda Inc.); p-isononylphenoxypoly-(glycidol), also known as Oline-10G or Surfactant™ 10-G (Olin Chemicals, Stamford, Conn.); Crodestas™ SL-40 (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)₄ ammonium chloride or bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT® 336), POLYQUAT™ 10, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL® and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such as benzalkonium chloride, a carbonium compound, a phosphonium compound, an oxonium compound, a halonium compound, a cationic organometallic compound, a quaternary phosphorous compound, a pyridinium compound, an anilinium compound, an ammonium compound, a hydroxylammonium compound, a primary ammonium compound, a secondary ammonium compound, a tertiary ammonium compound, and quaternary ammonium compounds of the formula NR₁R₂R₃R₄(O)⁺. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

### 2. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH 101 and Avicel® PH102, microcrystalline cellulose, and silicified microcrystalline cellulose (ProSolv SMCC®).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

### 3. Nanoparticulate Zafirlukast

The compositions of the invention contain nanoparticulate zafirlukast particles, which have an effective average particle size of less than about 2,000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 mm, less than about 75 nm, or less than about 50 nm, as measured by light-scattering methods, microscopy, or other appropriate methods.

By “an effective average particle size of less than about 2,000 nm” it is meant that at least 50% of the heterocyclic amide derivative, preferably zafirlukast particles have a particle size of less than the effective average, by weight, i.e., less than about 2,000 nm, 1900 nm, 1800 nm, etc., when measured by the above-noted techniques. Preferably, at least about 70%, about 90%, or about 95% of the heterocyclic amide derivative, and preferably zafirlukast particles, have a particle size of less than the effective average, i.e., less than about 2,000 nm, 1900 nm, 1800 nm, 1700 nm, etc.

In the present invention, the value for D50 of a nanoparticulate heterocyclic amide derivative and preferably zafirlukast composition is the particle size below which 50% of the heterocyclic amide derivative, and most preferably, zafirlukast particles fall, by weight. Similarly, D90 is the particle size below which 90% of the heterocyclic amide derivative, and most preferably, zafirlukast particles fall, by weight.

### 4. Concentration of the Heterocyclic Amide Derivatives and Surface Stabilizers

The relative amounts of heterocyclic amide derivative, and preferably zafirlukast, and one or more surface stabilizers can vary widely. The optimal amount of the individual components can depend, for example, upon the particular heterocyclic amide derivative selected, the hydrophilic lipophilic balance (HLB), melting point, and the surface tension of water solutions of the stabilizer, etc.

The concentration of the heterocyclic amide derivative, preferably zafirlukast, can vary from about 99.5% to about 0.001%, from about 95% to about 0.1%, or from about 90% to about 0.5%, by weight, based on the total combined weight of the zafirlukast and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the zafirlukast and at least one surface stabilizer, not including other excipients.

### 5. Exemplary Nanoparticulate Zafirlukast Tablet Formulations

Several potential exemplary zafirlukast tablet formulations are given below. These examples are not intended to limit the claims in any respect, but rather provide exemplary tablet formulations of heterocyclic amide derivative, and most preferably, zafirlukast, which can be utilized in the methods of the invention. Such exemplary tablets can also comprise a coating agent.

6.

Exemplary Nanoparticulate Zafirlukast Tablet Formulation #1

| Component | g/Kg |
|---|---|
| Zafirlukast | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrage, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate Zafirlukast Tablet Formulation #2

| Component | g/KG |
|---|---|
| Zafirlukast | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate Zafirlukast Tablet Formulations #3

| Component | g/Kg |
|---|---|
| Zafirlukast | about 200 to about 225 |
| Hypromellose, USP | about 42 to about 46 |
| Ducosate Sodium, USP | about 2 to about 6 |
| Sucrose, NF | about 200 to about 225 |
| Sodium Lauryl Sulfate, NF | about 12 to about 18 |
| Lactose Monohydrage, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate Zafirlukast Tablet Formulations #4

| Component | g/KG |
|---|---|
| Zafirlukast | about 119 to about 224 |
| Hypromellose, USP | about 42 to about 46 |
| Ducosate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

### C. Methods of Making Nanoparticulate Zafirlukast Compositions

The nanoparticulate heterocyclic amide derivative, preferably zafirlukast compositions can be made using, for example, milling, homogenization, or precipitation techniques or supercritical fluid techniques. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate zafirlukast compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

### 1. Milling to Obtain Nanoparticulate Zafirlukast Dispersions

Milling an heterocyclic amide derivative, preferably zafirlukast, to obtain a nanoparticulate dispersion comprises dispersing the zafirlukast particles in a liquid dispersion medium in which the zafirlukast is poorly soluble, followed by applying mechanical means in the presence of grinding media to reduce the particle size of the zafirlukast to the desired effective average particle size. The dispersion medium can be, for example, water, safflower oil, ethanol, t-butanol, glycerin, polyethylene glycol (PEG), hexane, or glycol. A preferred dispersion medium is water.

The heterocyclic amide derivative and preferably zafirlukast particles can be reduced in size in the presence of at least one surface stabilizer. Alternatively, the heterocyclic amide derivative, and most preferably, zafirlukast particles can be contacted with one or more surface stabilizers after attrition. Other compounds, such as a diluent, can be added to the zafirlukast/surface stabilizer composition during the size reduction process. Dispersions can be manufactured continuously or in a batch mode.

### 2. Precipitation to Obtain Nanoparticulate Zafirlukast Compositions

Another method of forming the desired nanoparticulate heterocyclic amide derivative derivatives, preferably zafirlukast, composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving zafirlukast in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

### 3. Homogenization to Obtain Nanoparticulate Zafirlukast Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of zafirlukast, in a liquid dispersion medium, followed by subjecting the dispersion to homogenization to reduce the particle size of the zafirlukast to the desired effective average particle size. The zafirlukast particles can be reduced in size in the presence of at least one surface stabilizer. Alternatively, the zafirlukast particles can be contacted with one or more surface stabilizers either before or after attrition. Other compounds, such as a diluent, can be added to the zafirlukast/surface stabilizer composition either before, during, or after the size reduction process. Dispersions can be manufactured continuously or in a batch mode.

### 4. Supercritical Fluid Techniques Used to Obtain Nanoparticulate Zafirlukast Compositions

Published International Patent Application No. WO 97/144407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers.

### D. Methods of Using the Zafirlukast Compositions of the Invention

The invention provides a method of rapidly increasing the plasma levels of zafirlukast in a subject. Such a method comprises orally administering to a subject an effective amount of a composition comprising nanoparticulate zafirlukast. The zafirlukast composition, in accordance with standard pharmacokinetic practice, produces a maximum blood plasma concentration profile in less than about 6 hours, less than about 5 hours, less than about 4 hours, less than about 3 hours, less than about 2 hours, less than about 1 hour, or less than about 30 minutes after the initial dose of the composition.

The compositions of the invention are useful in all treatments requiring zafirlukast, including but not limited to the treatment of asthma.

The zafirlukast compositions of the invention can be administered to a subject by any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms “patient” and “subject” may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate heterocyclic amide derivative, and preferably zafirlukast, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the zafirlukast, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

“Therapeutically effective amount” as used herein with respect to a zafirlukast, shall mean that dosage amount that provides the specific pharmacological response for which the zafirlukast is administered in a significant number of subjects in need of treatment for asthma and related disorders. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that zafirlukast dosages are, in particular instances, measured as oral dosages, or with reference to drug levels as measured in blood.

One of ordinary skill will appreciate that effective amounts of zafirlukast can be determined empirically and can be employed in pure form or, where such forms exist, in pharmaceutically acceptable salt, ester, or prodrug form. Actual dosage levels of zafirlukast in the nanoparticulate compositions of the invention may be varied to obtain an amount of the zafirlukast that is effective to obtain a desired therapeutic response for a particular composition and method of administration. The selected dosage level therefore depends upon the desired therapeutic effect, the route of administration, the potency of the administered zafirlukast, the desired duration of treatment, and other factors.

Dosage unit compositions may contain such amounts of such sub-multiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### Plasma Profile

The plasma profile associated with the administration of a drug compound may be described as a “pulsatile profile” in which pulses of high concentration heterocyclic amide derivative nanoparticles, preferably zafirlukast nanoparticles, interspersed with low concentration troughs, are observed. A pulsatile profile containing two peaks may be described as “bimodal.” Similarly, a composition or a dosage form which produces such a profile upon administration may be said to exhibit “pulsed release” of the zafirlukast.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash-out period provided by the fall off of the plasma concentration of the zafirlukast between peaks has been thought to be a contributing factor in reducing or preventing patient tolerance to various types of drugs.

Because the plasma profile produced by the controlled release composition upon administration is substantially similar to the plasma profile produced by the administration of two or more IR dosage forms given sequentially, the controlled release composition of the present invention is particularly useful for administering zafirlukasts for which patient tolerance may be problematical. This controlled release composition is therefore advantageous for reducing or minimizing the development of patient tolerance to the active ingredient in the composition. In the present invention, the heterocyclic amide derivative, preferably zafirlukast, and the controlled release composition in operation delivers the zafirlukast in a bimodal or pulsed manner.

Such a composition in operation produces a plasma profile which substantially mimics that obtained by the sequential administration of two IR doses as, for instance, in a typical zafirlukast treatment regime.

The present invention also provides solid oral dosage forms comprising a composition according to the invention. The present invention further provides a method of treating a patient suffering from asthma utilizing zafirlukast comprising administering a therapeutically effective amount of a composition or solid oral dosage form according to the invention to provide pulsed or bimodal administration of the zafirlukast. Advantages of the present invention include reducing the dosing frequency required by conventional multiple IR dosage regimes while still maintaining the benefits derived from a pulsatile plasma profile. This reduced dosing frequency is advantageous in terms of patient compliance to have a formulation which may be administered at reduced frequency. The reduction in dosage frequency made possible by utilizing the present invention would contribute to reducing health care costs by reducing the amount of time spent by health care workers on the administration of drugs.

### DEFINITIONS

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “controlled release” as used herein in relation to the composition according to the invention or used in any other context means release of nanoparticulate heterocyclic amide derivatives, and preferably zafirlukast nanoparticles, over time, and is taken to encompass sustained release and delayed release.

The term “time delay” as used herein refers to the duration of time between administration of the composition and the release of the heterocyclic amide derivative, and preferably zafirlukast, from a particular component.

The term “lag time” as used herein refers to the time between delivery of heterocyclic amide derivative, preferably zafirlukast, from one component and the second or subsequent component or formulation.

Heterocyclic amide derivatives and zafirlukast are collectively referred to herein as “active ingredients.” The active ingredient in each component may be the same or different. For example, a composition in which the first component comprises zafirlukast and the second component comprises zafirlukast in combination with a second ingredient effective in treating asthma may be desirable for combination therapies. Indeed, two or more heterocyclic amide derivatives may be incorporated into the same component when such active ingredients are compatible with each other.

### Additives

The heterocyclic amide derivative, and preferably zafirlukast, present in one component of the composition may be accompanied by, for example, an enhancer compound or a sensitizer compound in another component of the composition, in order to modify the bioavailability or therapeutic effect of the drug compound.

As used herein, the term “enhancer” refers to a compound which is capable of enhancing the absorption and/or bioavailability of an active ingredient by promoting net transport across the gastro-intestinal tract in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

### Proportion of Heterocyclic Amide Derivative and Additives

The proportion of the heterocyclic amide derivative, and preferably zafirlukast, contained in each component may be the same or different depending on the desired dosing regime. The heterocyclic amide derivative, and preferably zafirlukast, is present in the first component and in the second component in any amount sufficient to elicit a therapeutic response. The zafirlukast when applicable, may be present either in the form of one substantially optically pure enantiomer or as a mixture, racemic or otherwise, of enantiomers. The zafirlukast is preferably present in a composition in an amount of from 0.1-500 mg, preferably in the amount of from 1-100 mg. Zafirlukast is preferably present in the first component in an amount of from 0.5-60 mg; more preferably, the zafirlukast is present in the first component in an amount of from 2.5-30 mg. The zafirlukast is present in the subsequent components in an amount within a similar range to that described for the first component.

### Time Release Profile

The time release characteristics for the release of the nanoparticle heterocyclic amide derivative, preferably zafirlukast nanoparticles, from each of the components may be varied by modifying the composition of each component, including modifying any of the excipients or coatings which may be present. In particular, the release of zafirlukast may be controlled by changing the modified release constituent, including the amount of the modified release coating on the particles, if such a coating is present. As noted above, the time release profiles may be controlled by making the subsequent components or formulations in the form of erodable formulations, diffusion controlled formulations or osmotic controlled formulations. If more than one modified release component is present, the modified release coating for each of the subsequent components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time or delay time for the release of the nanoparticulate heterocyclic amide derivative, preferably zafirlukast nanoparticles, may also be varied by modifying the composition of each of the components, including modifying any excipients and coatings which may be present. For example, the first component may be an immediate release component wherein the zafirlukast is released substantially immediately upon administration. Alternatively, the first component may be, for example, a time-delayed immediate release component in which the zafirlukast is released substantially immediately after a time delay. The second component may be, for example, a time-delayed immediate release component as just described or, alternatively, a time-delayed sustained release or extended release component in which the zafirlukast is released in a controlled fashion for up to twenty-four hours.

### Plasma Concentration Curve

As will be appreciated by those skilled in the art, the exact nature of the plasma concentration curve will be influenced by the combination of all of these factors just described. In particular, the lag time between the delivery (and thus also the onset of action) of the heterocyclic amide derivative, and preferably, zafirlukast in each component may be controlled by varying the zafirlukast and coating (if present) of each of the components. Thus, by variation of each component (including the amount and nature of the zafirlukast) and by variation of the lag time, numerous release and plasma profiles may be obtained. Depending on the duration of the lag time between the release of zafirlukast from each component and the nature of the release constituent from each component (i.e., immediate release, sustained release etc.), the pulses in the plasma profile may be well separated and clearly defined peaks (e.g., when the lag time is long) or the pulses may be superimposed to a degree (e.g., in when the lag time is short).

In a preferred embodiment, the controlled release composition according to the present invention has a first immediate release component and at least one subsequent or modified release component. The immediate release component comprises a first population of active (i.e., heterocyclic amide derivative, preferably zafirlukast) ingredient-containing nanoparticles, and the modified release components or formulations comprise second and subsequent populations of active ingredient-containing nanoparticles. The second and subsequent modified release components or formulations may comprise a modified release coating. Additionally or alternatively, the second and subsequent modified release components may comprise a modified release matrix material. In operation, administration of such a modified release composition having, for example, a single modified release component, results in characteristic pulsatile plasma concentration levels of the zafirlukast in which the immediate release constituent of the composition gives rise to a first peak in the plasma profile and the modified release constituent gives rise to a second peak in the plasma profile. Embodiments of the invention comprising more than one modified release constituent give rise to further peaks in the plasma profile.

Such a plasma profile produced from the administration of a single dosage unit is advantageous when it is desirable to deliver two (or more) pulses of active ingredient without the need for administration of two (or more) dosage units. Additionally, in the case of asthma it is particularly useful to have such a bimodal plasma profile. For example, a typical zafirlukast treatment regime consists of administration of two doses of an immediate release dosage formulation given four hours apart. This type of regime has been found to be therapeutically effective and is widely used. As previously mentioned, the development of patient tolerance is an adverse effect sometimes associated with zafirlukast treatments. It is believed that the trough in the plasma profile between the two peak plasma concentrations is advantageous in reducing the development of patient tolerance by providing a period of wash-out of the zafirlukast. Drug delivery systems which provide zero order or pseudo zero order delivery of the zafirlukast do not facilitate this wash-out process.

### Modified Release Coating Material

Any coating material which modifies the release of the heterocyclic amine derivative, preferably zafirlukast, in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under EUDRAGIT® RS and RL, polyacrylic acid and poly acrylate and methacrylate copolymers such as those sold under the EUDRAGIT® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (EUDRAGIT® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. .about.5 k-5,000 k), polyvinylpyrrolidone (m. wt. .about.10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. .about.30 k-300 k), polysaccharides such as agar, acacia, karaya, tragacanth, algins and guar, polyacrylamides, POLYOX® polyethylene oxides (m. wt. .about.100 k-5,000 k), AQUAKEEP™ acrylate polymers, diesters of polyglucan, crosslinked polyvinyl alcohol and poly N-vinyl-2-pyrrolidone, sodium starch glucolate (e.g., EXPLOTAB®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g., Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g., EUDRAGIT®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticizers, lubricants, solvents and the like may be added to the coating. Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate and dibutyl sebacate.

### Modified Release Matrix Material

When the subsequent component or formulation comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of an heterocyclic amide derivative, preferably zafirlukast, dispersed therein in vitro or in vivo. Modified release matrix materials suitable for the practice of the present invention include but are not limited to microcrytalline cellulose, sodium carboxymethylcellulose, hydroxyalkylcelluloses such as hydroxypropyl-methylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acetate phthalate, cellulose acteate trimellitate, polyvinylacetate phthalate, polyalkylmethacrylates, polyvinyl acetate and mixtures thereof.

### Form of Dosage

A multiparticulate modified release composition according to the present invention may be incorporated into any suitable dosage form which facilitates release of the active ingredient in a pulsatile manner. Typically, the dosage form may be a blend of the different populations of heterocyclic amide derivative, preferably zafirlukast for the treatment of asthma. The zafirlukast-containing particles which make up the immediate release and the modified release components may be blended and the blend filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet. In this instance the first component of the controlled release composition may be compressed into one layer, with the second component being subsequently added as a second layer of the multilayer tablet. The populations of heterocyclic amide derivative, preferably zafirlukast containing nanoparticles making up the composition of the invention may further be included in rapidly dissolving dosage forms such as an effervescent dosage form or a fast-melt dosage form.

The composition according to the invention comprises at least two populations of heterocyclic amide derivative, preferably zafirlukast containing nanoparticles which have different in vitro dissolution profiles.

Preferably, in operation the composition of the invention and the solid oral dosage forms containing the composition release the zafirlukast such that substantially all of the zafirlukast contained in the first component is released prior to release of the zafirlukast from the second or subsequent component or formulation. When the first component comprises an IR component, for example, it is preferable that release of the zafirlukast from the second or subsequent component is delayed until substantially all the zafirlukast in the IR component has been released. Release of the zafirlukast from the second component may be delayed as detailed above by the use of a modified release coating and/or a modified release matrix material as part of erodable, diffusion controlled or osmotic controlled formulations.

More preferably, when it is desirable to minimize patient tolerance by providing a dosage regime which facilitates wash-out of a first dose of zafirlukast from a patient's system, release of the zafirlukast from the second component or formulation is delayed until substantially all of the zafirlukast contained in the first component has been released, and further delayed until at least a portion of the zafirlukast released from the first component has been cleared from the patient's system. In a preferred embodiment, release of the zafirlukast from the second component of the composition in operation is substantially, if not completely, delayed for a period of at least about two hours after administration of the composition and is released, preferably over the remaining twenty-four hour period after administration.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A stable nanoparticulate heterocyclic amide derivative composition comprising:
(a) zafirlukast particles having an effective average particle size of less than about 2,000 nm; and
(b) at least one surface stabilizer.

2. The composition of claim 1, wherein the zafirlukast is selected from the group consisting of a crystalline phase, an amorphous phase, a semi-crystalline phase, a semi-amorphous phase, and mixtures thereof.

3. The composition of claim 1, wherein the effective average particle size of the nanoparticulate zafirlukast particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1, wherein the composition is formulated for administration selected from the group consisting of oral, pulmonary, rectal, ocular, opthalmic, colonic, parenteral, intracistemal, intravaginal, intraperitoneal, local, buccal, nasal, and topical administration.

5. The composition of claim 1, wherein the composition further comprises one or more pharmaceutically acceptable excipients, carriers, or a combination thereof.

6. The composition of claim 1, wherein the zafirlukast is present in an amount selected from the group consisting of from about 99.5% to about 0.001%, from about 95% to about 0.1%, and from about 90% to about 0.5%, by weight, based on the total combined weight of the zafirlukast and at least one surface stabilizer, not including other excipients.

7. The composition of claim 1, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the zafirlukast and at least one surface stabilizer, not including other excipients.

8. The composition of claim 1, comprising at least one primary surface stabilizer and at least one secondary surface stabilizer.

9. The composition of claim 1, wherein the surface stabilizer is selected from the group consisting of an anionic surface stabilizer, a cationic surface stabilizer, a zwitterionic surface stabilizer, and an ionic surface stabilizer.

10. The composition of claim 9, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

11. The composition of claim 9, wherein the at least one cationic surface stabilizer is selected from the group consisting of a polymer, a biopolymer, a polysaccharide, a cellulosic, an alginate, a non-polymeric compound, and a phospholipid.

12. The composition of claim 9, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl (ethenoxy)₄ ammonium bromide, N-alkyl(C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride, dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, alkyl pyridinium salts; amines, amine salts, amine oxides, imide azolinium salts, protonated quaternary acrylamides, methylated quaternary polymers, and cationic guar.

13. The composition of claim 9, wherein the zafirlukast is bioadhesive.

14. The composition of claim 1, comprising hypomellose, dioctyl sodium sulfosuccinate, and sodium lauryl sulfate as surface stabilizers.

15. An asthma treatment comprising the following components:
(a) about 50 to about 500 g/kg zafirlukast;
(b) about 10 to about 70 g/kg hypromellose;
(c) about 1 to about 10 g/kg docusate sodium;
(d) about 100 to about 500 g/kg sucrose;
(e) about 1 to about 40 g/kg sodium lauryl sulfate;
(f) about 50 to about 400 g/kg lactose monohydrate;
(g) about 50 to about 300 g/kg silicified microcrystalline cellulose;
(h) about 20 to about 300 g/kg crospovidone; and
(i) about 0.5 to about 5 g/kg magnesium stearate.

16. The composition of claim 15, further comprising a coating agent.

17. An asthma treatment composition comprising the following components:
(a) about 100 to about 300 g/kg zafirlukast;
(b) about 30 to about 50 g/kg hypromellose;
(c) about 0.5 to about 10 g/kg docusate sodium;
(d) about 100 to about 300 g/kg sucrose;
(e) about 1 to about 30 g/kg sodium lauryl sulfate;
(f) about 100 to about 300 g/kg lactose monohydrate;
(g) about 50 to about 200 g/kg silicified microcrystalline cellulose;
(h) about 50 to about 200 g/kg crospovidone; and
(i) about 0.5 to about 5 g/kg magnesium stearate.

18. The composition of claim 17, further comprising a coating agent.

19. An asthma treatment composition comprising the following components:
(a) about 200 to about 225 g/kg zafirlukast;
(b) about 42 to about 46 g/kg hypromellose;
(c) about 2 to about 6 g/kg docusate sodium;
(d) about 200 to about 225 g/kg sucrose;
(e) about 12 to about 18 g/kg sodium lauryl sulfate;
(f) about 200 to about 205 g/kg lactose monohydrate;
(g) about 130 to about 135 g/kg silicified microcrystalline cellulose;
(h) about 112 to about 118 g/kg crospovidone; and
(i) about 0.5 to about 3 g/kg magnesium stearate.

20. The composition of claim 19, further comprising a coating agent.

21. An asthma treatment composition comprising the following components:
(a) about 119 to about 224 g/kg zafirlukast;
(b) about 42 to about 46 g/kg hypromellose;
(c) about 2 to about 6 g/kg docusate sodium;
(d) about 119 to about 224 g/kg sucrose;
(e) about 12 to about 18 g/kg sodium lauryl sulfate;
(f) about 119 to about 224 g/kg lactose monohydrate;
(g) about 129 to about 134 g/kg silicified microcrystalline cellulose;
(h) about 112 to about 118 g/kg crospovidone; and
(i) about 0.5 to about 3 g/kg magnesium stearate.

22. The composition of claim 21, further comprising a coating agent.

23. A stable nanoparticulate zafirlukast composition comprising:
(a) particles of a zafirlukast active or a salt thereof; and
(b) associated with the surface thereof dioctyl sodium sulfosuccinate and hypromellose;
wherein the zafirlukast particles have an effective average particle size of less than about 2,000 nm.

24. The composition of claim 23, further comprising sodium lauryl sulfate.

25. A method of making a nanoparticulate zafirlukast composition comprising contacting zafirlukast particles with at least one surface stabilizer for a time and under conditions sufficient to provide a nanoparticulate zafirlukast composition having an effective average particle size of less than about 2,000 nm.

26. The method of claim 25, wherein said contacting comprises grinding.

27. The method of claim 26, wherein said grinding comprises wet grinding.

28. The method of claim 25, wherein said contacting comprises homogenizing.

29. The method of claim 25, wherein said contacting comprises supercritical fluids processing.

30. The method of claim 25, wherein said contacting comprises:
(a) dissolving the zafirlukast particles in a solvent;
(b) adding the resulting zafirlukast solution to a solution comprising at least one surface stabilizer; and
(c) precipitating the solubilized zafirlukast having at least one surface stabilizer adsorbed on the surface thereof by the addition thereto of a non-solvent.

31. The method of claim 25, wherein the zafirlukast is selected from the group consisting of a crystalline phase, an amorphous phase, a semi-crystalline phase, a semi-amorphous phase, and mixtures thereof.

32. The method of claim 25, wherein the effective average particle size of the nanoparticulate zafirlukast particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

33. The method of claim 25, wherein the composition is formulated for administration selected from the group consisting of oral, pulmonary, rectal, ocular, colonic, parenteral, intracisternal, intravaginal, intraperitoneal, local, buccal, nasal, and topical administration.

34. The method of claim 25, wherein the composition further comprises one or more pharmaceutically acceptable excipients, carriers, or a combination thereof.

35. The method of claim 25, wherein the zafirlukast is present in an amount selected from the group consisting of from about 99.5% to about 0.001%, from about 95% to about 0.1%, and from about 90% to about 0.5%, by weight, based on the total combined weight of the zafirlukast and at least one surface stabilizer, not including other excipients.

36. The method of claim 25, wherein the at least one surface stabilizer is present in an amount selected from the group consisting of from about 0.5% to about 99.999%, from about 5.0% to about 99.9%, and from about 10% to about 99.5% by weight, based on the total combined dry weight of the zafirlukast and at least one surface stabilizer, not including other excipients.

37. The method of claim 25, comprising at least one primary surface stabilizer and at least one secondary surface stabilizer.

38. The method of claim 25, wherein the surface stabilizer is selected from the group consisting of an anionic surface stabilizer, a cationic surface stabilizer, a zwitterionic surface stabilizer, and an ionic surface stabilizer.

39. The method of claim 25, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

40. The method of claim 38, wherein the at least one cationic surface stabilizer is selected from the group consisting of a polymer, a biopolymer, a polysaccharide, a cellulosic, an alginate, a nonpolymeric compound, and a phospholipid.

41. The method of claim 25, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl (ethenoxy)₄ ammonium bromide, N-alkyl(C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride, dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, alkyl pyridinium salts; amines, amine salts, amine oxides, imide azolinium salts, protonated quaternary acrylamides, methylated quaternary polymers, and cationic guar.

42. The method of claim 38, wherein the zafirlukast composition is a bioadhesive.

43. The method of claim 25, wherein the composition comprises hypromellose, dioctyl sodium sulfosuccinate, and sodium lauryl sulfate as surface stabilizers.

44. A method for the treatment of asthma with a nanoparticulate zafirlukast comprising administering to the subject an effective amount of a nanoparticulate composition comprising particles of a zafirlukast having at least one surface stabilizer associated with the surface thereof, wherein the zafirlukast particles have an effective average particle size of less than about 2,000 nm.

45. The method of claim 44, wherein the zafirlukast is selected from the group consisting of a crystalline phase, an amorphous phase, a semi-crystalline phase, a semi-amorphous phase, and mixtures thereof.

46. The method of claim 44, wherein the effective average particle size of the nanoparticulate zafirlukast particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

47. The method of claim 44, wherein the composition is formulated for administration selected from the group consisting of oral, pulmonary, rectal, ocular, opthalmic, colonic, parenteral, intracistemal, intravaginal, intraperitoneal, local, buccal, nasal, and topical administration.

48. The method of claim 44, wherein the composition further comprises one or more pharmaceutically acceptable excipients, carriers, or a combination thereof.

49. The method of claim 44, wherein the zafirlukast is present in an amount selected from the group consisting of from about 99.5% to about 0.001%, from about 95% to about 0.1%, and from about 90% to about 0.5%, by weight, based on the total combined weight of the zafirlukast and at least one surface stabilizer, not including other excipients.

50. The method of claim 44, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the zafirlukast and at least one surface stabilizer, not including other excipients.

51. The method of claim 44, comprising at least one primary surface stabilizer and at least one secondary surface stabilizer.

52. The method of claim 44, wherein the surface stabilizer is selected from the group consisting of an anionic surface stabilizer, a cationic surface stabilizer, a zwitterionic surface stabilizer, and an ionic surface stabilizer.

53. The method of claim 50, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

54. The method of claim 52, wherein the cationic surface stabilizer is selected from the group consisting of a polymer, a biopolymer, a polysaccharide, a cellulosic, an alginate, a nonpolymeric compound, and a phospholipid.

55. The method of claim 44, wherein the surface stabilizer is selected from the group consisting of benzalkonium chloride, polymethylmethacrylate trimethylammonium bromide, polyvinylpyrrolidone-2-dimethylaminoethyl methacrylate dimethyl sulfate, hexadecyltrimethyl ammonium bromide, cationic lipids, sulfonium compounds, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride, dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, alkyl pyridinium salts; amines, amine salts, amine oxides, imide azolinium salts, protonated quaternary acrylamides, methylated quaternary polymers, and cationic guar.

56. The method of claim 52, wherein the zafirlukast composition is bioadhesive.

57. The method of claim 44, wherein the composition comprises hypromellose, dioctyl sodium sulfosuccinate, and sodium lauryl sulfate as surface stabilizers.

58. The method of claim 44, wherein the method is used for the treatment of asthma in a subject which is a mammal.

59. The method of claim 58, wherein said subject is a human.

60. The method of claim 44, wherein said composition is an oral suspension.

61. The method of claim 44, wherein said composition is a dosage form selected from the group consisting of liquid dispersions, gels, aerosols, ointments, creams, controlled release formulations, fast melt formulations, lyophilized formulations, tablets, capsules, delayed release formulations, extended release formulations, pulsatile release formulations, and mixed immediate release and controlled release formulations.

62. The method of claim 44, wherein the effective amount is 10 to 20 mg per day.

63. A controlled release composition consisting essentially of: (A) a first component comprising a first population of heterocyclic amide derivative nanoparticles; and (B) at least one subsequent component or formulation comprising a subsequent population of heterocyclic amide derivative nanoparticles and a modified release constituent comprising a modified release coating, a modified release matrix material or mixtures thereof; wherein the composition, following oral delivery to a subject, delivers the heterocyclic amide derivative nanoparticles in the first and subsequent populations in a pulsatile manner.

64. The composition of claim 63, wherein the heterocyclic amide derivative in the first and subsequent populations is zafirlukast and said modified release constituent delivers to a subject the subsequent population of zafirlukast over a period of up to twenty-four hours after administration.

65. The composition according to claim 64, comprising a modified release coating.

66. The composition according to claim 63, wherein the first population comprises immediate-release particles and the formulation comprising the subsequent population is an erodable formulation.

67. The composition according to claim 63, wherein the formulation comprising the subsequent population is a diffusion controlled formulation.

68. The composition according to claim 63, wherein the formulation comprising the subsequent population is an osmotic controlled formulation.

69. The composition of claim 63, wherein the formulation comprises a modified release matrix material.

70. The composition according to claim 69, wherein the composition further comprises an enhancer.

71. The composition according to claim 70, wherein the amount of zafirlukast contained in each of the first and subsequent populations is from about 10 mg to about 20 mg.

72. The composition according to claim 64, wherein the first and subsequent populations have different in vitro dissolution profiles.

73. The composition according to claim 72, which in operation releases substantially all of the zafirlukast from the first population prior to release of the zafirlukast nanoparticles from the subsequent population.

74. The composition according to claim 64, comprising a blend of the particles of each of the first and subsequent populations contained in a hard gelatin or soft gelatin capsule.

75. The composition according to claim 64, wherein the particles of each of the populations are in the form of mini-tablets and the capsule contains a mixture of the mini-tablets.

76. The composition according to claim 64, in the form of a multilayer tablet comprising a first layer of compressed zafirlukast nanoparticles of the first population and another layer of zafirlukast-containing particles of the subsequent population

77. The composition according to claim 76, wherein the first and subsequent populations of zafirlukast-containing nanoparticles are provided in a rapidly dissolving dosage form.

78. The composition according to claim 77, wherein the particles of each of the populations are compressed into a fast-melt tablet.

79. A method for the treatment of asthma comprising administering a therapeutically effective amount of a composition according to claim 64.

80. The composition according to claim 64, wherein the subsequent formulation comprises a pH-dependent polymer coating which is effective in releasing a pulse of the active ingredient following a time delay.

81. The composition according to claim 80, wherein the polymer coating comprises methacrylate copolymers.

82. The composition according to claim 81, wherein the polymer coating comprises a mixture of methacrylate and ammonio methacrylate copolymers in a ratio sufficient to achieve a pulse of the active ingredient following a time delay.

83. The composition according to claim 82, wherein the ratio of methacrylate to ammonio methacrylate copolymers is 1:1.
